(19) United States
(12) Patent Application Publication
(10) Pub. No.: US 2025/0311765 A1
(43) Pub. Date: Oct. 9, 2025

(54) POUCHES COMPRISING ACTIVE INGREDIENTS

(71) Applicant: ODS Products, LLC, Austin, TX (US)

(72) Inventor: Samuel BENAIM, Newport Beach, CA (US)

(73) Assignee: ODS Products, LLC, Austin, TX (US)

(21) Appl. No.: 19/197,683

(22) Filed: May 2, 2025

Related U.S. Application Data
(63) Continuation of application No. PCT/US2025/023123, filed on Apr. 4, 2025.
(60) Provisional application No. 63/575,393, filed on Apr. 5, 2024.
(60) Provisional application No. 63/683,993, filed on Aug. 16, 2024.

(51) Int. Cl. A24B 15/24; A24B 15/16; A24B 15/42; A24F 23/02

(52) U.S. Cl. CPC A24B 15/243; A24B 15/165; A24B 15/42; A24F 23/02

(57) ABSTRACT

Pouch bases systems for the oral delivery of an active are provided. These systems may have quick dissolution rates which may be created by leveraging the use of high-viscosity and/or low-viscosity polymers in spheronized extrudates. These systems may also comprise a plurality of different particles which may enhance therapeutic and/or sensorial effect of the administration systems. Methods of producing these systems are also provided.

### CROSS-REFERENCE TO RELATED APPLICATIONS

The present application is a continuation of PCT/US2025/023123, filed Apr. 4, 2025, which claims priority to and the benefit of U.S. App. No. 63/575,393, filed Apr. 5, 2024, and U.S. App. No. 63/683,993, filed Aug. 16, 2024, each of which are hereby incorporated by reference in their entirety.

### FIELD OF DISCLOSURE

The present disclosure is related to pouches containing spheronized extrudate particles comprising an active agent such as nicotine and/or caffeine and methods of manufacture thereof.

### BACKGROUND

Traditional means of orally administering actives such as nicotine involve the use of materials that can become sticky and feel unpleasant in the mouth during use. Many nicotine products are available in small containers called pouches. These pouches generally are made of saliva permeable material containing a material having the active therein. Pouches are often plagued by design issues such as unstable particle size and active delivery systems. Moreover, limitations on these materials used often limit the incorporation of various materials, such as flavorants, providing limited access to both excipient and active formulation for formulators.

The release profile of the active is also important to oral pouch delivery systems. Generally, these systems operate via saliva entering the cavity of the pouch dissolving or extracting some amount of active, to then migrate through the pouch for oral delivery. The material in the pouches needs to be released in a controlled manner to achieve the appropriate therapeutic and/or sensorial effect a formulator would want a user to experience. Increasing the ability to alter the release profile, possibly customized to and for the incorporated active, or for a sensorial quality such as taste or mouth feel, would offer wider access to formulators to diversify and create pouches having better therapeutic and/or sensorial effect.

It is therefore an object of this disclosure to pouches and methods of making pouches having different release profiles from those that exist and which offer a wider variety of therapeutic and/or sensorial effect.

### SUMMARY

In accordance with the foregoing objectives and others, the present disclosure provides pouches comprising an active (e.g., nicotine and salts and cyclodextrin forms thereof, IMOTINE® (6-methylnicotine) and salts and cyclodextrin forms thereof, caffeine, lidocaine, synephrine, kratom such as Mitragyna speciosa extracts or any alkaloids present therein such as mitragynine) incorporated into a plurality of particles. Typically, the active is incorporated into the core of the particle which may be optionally coated. The core may comprise the active and a filler or binder such as Gum Arabic, Methyl Cellulose, Liquid glucose, Tragacanth, Ethyl Cellulose, Gelatin, Hydroxy Propyl Methyl Cellulose (HPMC), Starches (natural or modified), Hydroxy Propyl Cellulose (HPC), Pregelatinized Starch, Sodium Carboxy Methyl Cellulose (NaCMC), Alginic Acid, Polyvinyl Pyrrolidone (PVP), Maltodextrin (MD); Cellulose, Polyethylene Glycol (PEG), Polyvinyl Alcohols, Polymethacrylates, Copovidone or Microcrystalline Cellulose (MCC), alone or in combination. In some embodiments, the plurality of particles in the pouch is a homogenous mixture of particles (e.g., more than 95% or more than 99% or more than 99.5% of the particles by weight of the plurality of particles have cores formed from the same extrudate, more than 95% or more than 99% or more than 99.5% of the particles by weight of the plurality of particles have cores from the same extrudate and are coated with the same coating).

The plurality of particles may comprise two different pluralities of particles having at least one different characteristic such as color (e.g., the different pluralities have a different color), taste (e.g., the different pluralities have a different taste), active (e.g., the different pluralities have different actives or one plurality does not contain an active), particle size (e.g., the different pluralities have different particle size). For example, in some embodiments, the pouches comprise a first plurality of particles and a second plurality of particles, wherein the first plurality particles and the second plurality of particles have a difference in the CIE L*a*b* color space (e.g., a ΔE greater than 3 or greater than 5 or greater than 10 or greater than 20 or greater than 50). In some embodiments, one of the first plurality of particles or the second plurality of particles comprises a colorant (e.g., in the core, in the coating) and the other of the first plurality of particles or the second plurality of particles does not comprise a colorant (or less than 95% or 99% or 99.5% by weight colorant). The sensorial and/or therapeutic benefit of the presently disclosed systems may be increased through the use of different pluralities of particles in the pouch. These particles may have at least one characteristic distinct from one another (e.g., particle size, color, flavor, active, binder, excipient, release profile). In particular embodiments, the first plurality of particles and the second plurality of particles comprise the same low viscosity polymer (e.g., in a weight percentage of low viscosity polymer within 10% of one another).

A system is provided (e.g., for oral delivery of an active) comprising:
a) a pouch; and
b) a plurality of particles contained within the pouch;
wherein, the particles comprise an active, a filler, a buffer, a low viscosity polymer, and a high viscosity polymer. In some embodiments, the plurality of particles is a homogenous mixture. In various implementations the plurality of particles comprises a core and optionally a coating, wherein the cores of the plurality of particles are formed from the same extrudate. Without wishing to be bound by theory, the present disclosure is also partially premised on the discovery that altering the content of specific viscosity polymers (e.g., low-viscosity polymer, high viscosity polymer) affords control of the release profile of any actives present in spheronized particles. In particular, the low-viscosity polymer to high-viscosity polymer ratio may afford faster release of active from any particles contained in the pouch. In some embodiments, the weight ratio of low viscosity polymer to high viscosity polymer is from 100:1 to 1:100 (e.g., 100:1 to 1:1, 50:1 to 1:1, 30:1 to 1:1, from 5:1 to 1:1, from 10:1 to 5:1, from 15:1 to 10:1 from 20:1 to 15:1 from 25:1 to 20:1, from 30:1 to 25:1, from 9:1 to 1:9). In some embodiments, more than 45% (e.g., more than 50%, more than 60%) by weight of the active is released from the plurality of particles or the first plurality of particles or the second plurality of particles within 5 minutes in 500 mL H₂O at 37° C. and spinning via a paddle at 50 rpm (e.g., as measured by 260 nm spectroscopy). In some embodiments, less than 50% (e.g., less than 40%, less than 30%) by weight of the active is released from the plurality of particles or the first plurality of particles or the second plurality of particles within 5 minutes in 500 mL H₂O at 37° C. and spinning via a paddle at 50 rpm (e.g., as measured by 260 nm spectroscopy). In some embodiments, the spheres may comprise a combination of polymers of similar viscosity (e.g., a combination of low viscosity polymers such as modified starch and hydroxypropylmethylcellulsoe in a, for example, a weight ratio of 100:1 to 1:1 (e.g., 50:1 to 1:1, 25:1 to 1:1, 20:1 to 2:1, 50:1 to 2:1, 25:1 to 2:1, 20:1 to 2:1).

In some embodiments, the plurality of particles comprises a first plurality of particles and a second plurality of particles, wherein the first and second pluralities of particles have at least one characteristic distinct from one another (e.g., particle size, color, flavor, active). In various implementations, the first and/or second plurality of particles comprises a colorant, a flavoring agent, a sweetener, or a combination thereof. In some embodiments, the first plurality of particles contains a first active (e.g., nicotine or pharmaceutically acceptable salts and/or cyclodextrin forms thereof, polacrilex, IMOTINE® (6-methylnicotine) or pharmaceutically acceptable salts and/or cyclodextrin forms thereof, lidocaine, synephrine, kratom such as Mitragyna speciosa extracts or any alkaloids present therein such as mitragynine) and the second plurality of particles contains a second active (e.g., caffeine or pharmaceutically acceptable salts thereof). In some embodiments, the first plurality of particles comprises a first core (e.g., comprising a filler or binder such as Gum Arabic, Methyl Cellulose, Liquid glucose, Tragacanth, Ethyl Cellulose, Gelatin, Hydroxy Propyl Methyl Cellulose (HPMC), Starches (natural or modified), Hydroxy Propyl Cellulose (HPC), Pregelatinized Starch, Sodium Carboxy Methyl Cellulose (NaCMC), Alginic Acid, Polyvinyl Pyrrolidone (PVP), Maltodextrin (MD); Cellulose, Polyethylene Glycol (PEG), Polyvinyl Alcohols, Polymethacrylates, Copovidone or Microcrystalline Cellulose (MCC), alone or in combination) and a first coating on that core (e.g., a coating comprising a saliva-soluble binder such as hydroxypropyl methylcellulose (HPMC), hydroxypropyl cellulose (HPC), methyl cellulose (MC), polyvinylpyrrolidone and mixtures thereof). According to an embodiment of the invention, the one or more binders comprises one or more cellulose binders. In an embodiment of the invention the one or more binders comprises microcrystalline cellulose (MCC), hydroxypropyl cellulose (HPC) or hydroxypropylmethyl cellulose (HPMC) or any combination thereof. In certain aspects, the first plurality of particles does not contain a colorant and the second plurality of particles contains a colorant. In various embodiments, the weight ratio of the first plurality of particles to the second plurality of particles is from 100:1 to 1:100 (e.g., 50:1 to 1:50, 25:1 to 1:25, 10:1 to 1:10, 100:1 to 50:1, 50:1 to 25:1, 25:1 to 10:1, 10:1 to 1:1, 1:1 to 1:10, 1:10 to 1:25, 1:25 to 1:50, 1:50 to 1:100). In particular embodiments, the filler comprises (or is) microcrystalline cellulose. In some aspects, the buffer comprises sodium phosphate (e.g., disodium phosphate), sodium carbonate, sodium citrate (e.g., icton), sodium bicarbonate, or a combination thereof. In some embodiments, the low viscosity polymer and/or the high viscosity polymer comprises a modified or natural starch (e.g., optionally modified corn starch, optionally modified wheat starch, optionally modified potato starch, optionally modified arrowroot starch, optionally modified cassava starch, optionally modified chitosan, optionally modified sodium alginate). In some embodiments, the low viscosity polymer is selected from modified corn starch, modified wheat starch, modified tapioca starch, modified potato starch, polyvinylpyrrolidone (PVP), hydroxypropyl methyl cellulose (HPMC), hydroxypropyl cellulose (HPC), or combinations thereof. In various implementations, the high viscosity polymer is selected from modified corn starch, modified wheat starch, modified tapioca starch, modified potato starch, hydroxypropyl methyl cellulose (HPMC), or combinations thereof.

In some embodiments, the particles in the first plurality of particles comprise a first core and a first coating. In some embodiments, the particles in the second plurality of particles comprise a second core and a second coating. Various components may be integrated into these coatings and cores and provide systems providing unique sensorial application. For example the first plurality of particles may have an active integrated into the core, while the second plurality of particles may have an active and/or flavorant integrated into the coating. Such configurations may allow a formulator to tailor the resultant system to provide different release profiles for different components (e.g., actives, flavorants) resultant in different sensations for the product. For example, in some embodiments, the first plurality of particles may comprise nicotine or imotine having immediate release, while the second plurality of particles may comprise caffeine having sustained or delayed release. In some embodiments, an active in the first plurality of particles (e.g., as present in a core and/or coating of the first plurality of particles) has a different release profile than an active and/or flavorant in the second plurality of particles (e.g., as present in a core and/or coating of the second plurality of particles).

The high-viscosity or low-viscosity polymers may be characterized by having specific viscosities. For example, a 2% by weight aqueous solution of the low viscosity polymer may have a viscosity of less than (or from 1 cP to) 50 cP (e.g., less than 20 cP, less than 10 cp) at 25° C. In certain embodiments, a 30% by weight aqueous solution of the low viscosity polymer (e.g., modified corn starch) has a viscosity of less than (or from 1 cP to) 50 cP (e.g., less than 20 cP, less than 10 cp, less than 5 cP) at 25° C. In some embodiments, a 2% by weight aqueous solution of the high viscosity polymer has a viscosity of more than (or up to 200,000 cP) 50 cP (e.g., more than 100 cP, from 100 cP to 100,000 cp) at 25° C. In some embodiments, the relative viscosity ratio of the high viscosity polymer to low viscosity polymer at 25° C. is from 100:1 to 200:1 (e.g., from 125:1 to 175:1) as produced from, for example, a modified starch polymer.

At least one particle in the plurality of particles (e.g., at least one particle in the first plurality of particles, at least one particle in the second plurality of particles, more than 95% or more than 99% by weight of all particles in the first plurality of particles, more than 95% or more than 99% by weight of all the particles in the second plurality of particles, more than 95% or more than 99% by weight of all the particles in the plurality of particles, all particles in the first plurality of particles, all particles in the second plurality of particles, all particles in the plurality of particles) may independently comprise one or more of:
a) 30%-80% (e.g., 30%-40%, 40%-50%, 50%-60%, 60%-70%, 70%-80%, 30%-65%) binder (e.g., microcrystalline cellulose) by weight of the particle;
b) optionally 5%-30% (e.g., 5-25%) flavorant and/or sweetener (e.g., plated on the binder) by weight of the particle;
c) 1%-10% (e.g., 10-20% buffer (e.g., disodium phosphate, sodium carbonate, sodium bicarbonate, trisodium phosphate, and combinations thereof) by weight of the particle;
d) 1%-10% (e.g., 5-10%) low viscosity polymer (e.g., HPMC, modified starch such as OSA-starch, combinations thereof) by weight of the particle;
e) optionally 0.5-5% (e.g., 0.5-1%) high viscosity polymer (e.g., modified starch such as OSA-starch) by weight of the particle;
f) 0.1-10% (e.g., 1-5%) active (e.g., nicotine), or active in an inclusion complex thereof (e.g., B-cyclodextrin/nicotine), by weight of the particle;
g) optionally less than (or from 0.1% to) 5% (e.g., less than 1%) colorant by weight of the particle; and
h) optionally less than (or from 0.01% to) 10% or less than 5% or less than 1% coating by weight of the particle.

The particles (or a subset of the plurality such as the first plurality of particles or the second plurality of particles) may comprise a colorant, and the colorant comprises a lake (e.g., FD&C Blue Lake #1 Lake; FD&C Blue Lake #2; FD&C Yellow #5 Lake; FD&C Yellow #6 Lake; FD&C Red #40 Lake) and/or a natural pigment (e.g., beet, turmeric, vitamin A). In various aspects, at least one particle in the plurality of particles comprises (e.g., at least one particle in the first plurality of particles, at least one particle in the second plurality of particles, more than 95% or more than 99% by weight of all particles in the first plurality of particles, more than 95% or more than 99% by weight of all the particles in the second plurality of particles, more than 95% or more than 99% by weight of all the particles in the plurality of particles, all particles in the first plurality of particles, all particles in the second plurality of particles, all particles in the plurality of particles) comprises a flavorant selected from watermelon flavor, citrus flavor, or wintergreen flavor.

The pouch may be transparent or translucent. Transparent or translucent pouches are particularly useful in embodiments comprising a colorant or different particle sizes in each plurality. In some embodiments, the weight of the plurality of particles in the pouch is less than 5 g (e.g., less than 2 g, less than 1.5 g, less than 1 g, from 400 mg to 600 mg, from 450 mg to 550 mg, from 100 mg to 5 g, from, 100 mg to 2 g, from 100 mg to 1.5 g, from 100 mg to 1 g, from 1 g to 2 g, less than 1.5 g, less than 1 g, from 400 mg to 600 mg, from 450 mg to 550 mg). In some embodiments, the pouch has an outer diameter (or largest dimension) of more than (or up to 100 mm) 20 mm (e.g., more than 25 mm, from 20 mm to 40 mm). In various aspects, the pouch is from 20 mm to 40 mm long and from 10-20 mm wide.

In specific aspects, the plurality of particles (or a subset thereof such as the first plurality of particles or the second plurality of particles) is uncoated. In some embodiments the first and/or second plurality of particles comprising a coating such as a shellac or carnauba wax coating. In some embodiments, the coating consists of carnauba wax and/or shellac. In various implementations, the coating may further comprise an active and/or buffer and/or polymer and/or flavorant and/or colorant such as a pigment. In various implementations, the coating does not contain an active. In some embodiments, the coating contains an active. In various implementations, none of the particles are coated with a source of nicotine. In certain aspects, less than 50% or less than 40% or less than 30% or less than 20% or less than 10% of the particles in the system are coated with a source of nicotine.

Systems are provided comprising:
a) a pouch; and
b) a plurality of particles comprising an active and contained within the pouch;
wherein, at least a portion of the particles comprise a colorant. The plurality of particles may comprise a first plurality of particles and a second plurality of particles, wherein the first and second pluralities of particles have at least one characteristic distinct from one another (e.g., particle size, color, flavor, active). In some embodiments, the first and/or second plurality of particles comprises a colorant, a flavoring agent, a sweetener, or a combination thereof. In some embodiments, the first plurality of particles contains a first active (e.g., nicotine or pharmaceutically acceptable salts and/or cyclodextrin forms thereof, polacrilex, IMOTINE® (6-methylnicotine) or pharmaceutically acceptable salts and/or cyclodextrin forms thereof, lidocaine, synephrine, kratom such as Mitragyna speciosa extracts or any alkaloids present therein such as mitragynine) and the second plurality of particles contains a second active (e.g., caffeine or pharmaceutically acceptable salts thereof, lidocaine, synephrine, kratom such as Mitragyna speciosa extracts or any alkaloids present therein such as mitragynine) and the second plurality of particles contains a second active (e.g., caffeine or pharmaceutically acceptable salts thereof). In some embodiments, the first plurality of particles does not contain a colorant and the second plurality of particles contains a colorant. In various aspects, the weight ratio of the first plurality of particles to the second plurality of particles is from 100:1 to 1:100 (e.g., 50:1 to 1:50, 25:1 to 1:25, 10:1 to 1:10, 100:1 to 50:1, 50:1 to 25:1, 25:1 to 10:1, 10:1 to 1:1, 1:1 to 1:10, 1:10 to 1:25, 1:25 to 1:50, 1:50 to 1:100).

Methods for producing the pluralities of particles are also provided generally involving mixing dry materials, spraying with an active solution, extruding and spheronizing to form a plurality of particles. The method may comprise:
a) mixing a binder, a low viscosity polymer and, optionally, a colorant and/or high viscosity polymer to form a mixture;
b) spraying a liquid composition (e.g., a composition comprising an active) onto the mixture or during a portion of the mixing step;
c) extruding the sprayed mixture to form an extrudate;
d) spheronizing the extrudate to form a spheronized extrudate;
wherein an active is mixed with the binder or present in the liquid composition.

The method may further comprise:
e) heating the spheronized extrudate (e.g., to dry to less than 3% by weight moisture measured on a moisture balance).

The heating may occur at a temperature having an intake heat of from 110° C.-130° C. and an exhaust heat of from 65-85° C. (e.g., from 70-78° C.). Moisture balance may be identified, for example, at 130° F. (such as with an Ohaus Model MB27).

The liquid composition may comprise the active (e.g., nicotine) dissolved in an solvent such as a mixture of water and humectant such as propylene glycol or glycerol. In various aspects, following spraying (e.g., prior to said extruding) with the liquid composition, the sprayed mixture is further mixed (e.g., for a time period of from 10 seconds to 1 minute). In some embodiments, the active (e.g., caffeine) is mixed in step a) with the dry ingredients.

Extruding may be performed in a suitable extruder. The mixture (e.g., sprayed mixture) may be extruded through a die having from 0.3-2 mm holes (e.g., from 0.5-1.5 mm, from 1-1.5 mm)). The extruder may be operated at from 10-1800 rpm (e.g., 10-600 rpm).

The extruded particle matter may be selected for a specific particle size. For example, the method may further comprise;
f) screening the spheronized extrudate (e.g., by passing through a first mesh and being collected in a second mesh); and/or
g) placing the spheronized extrudate in a pouch.

These methods may involve mixing pluralities of particles prepared from different batches (e.g., mixing a first plurality of particles with a second plurality of particles). The method may further comprise mixing the spheronized extrudate with a second spheronized extrudate having at least one characteristic distinct from the spheronized extrudate (e.g., particle size, color, flavor, active) to form a mixed spheronized extrudate and optionally placing the mixed spheronized extrudate in a pouch. In some embodiments, the comprises forming the second spheronized extrudate. For example, forming the second spheronized extrudate comprises
a1) mixing a binder, a low viscosity polymer and, optionally, a colorant and/or high viscosity polymer to form a second mixture;
b1) spraying a second liquid composition comprising an active onto the second mixture or during a portion of the mixing step;
c1) extruding the sprayed mixture to form a second extrudate;
d1) spheronizing the extrudate to form the second spheronized extrudate.

In various aspects, forming the second spheronized extrudate further comprises:
e1) heating the second spheronized extrudate (e.g., to dry to less than 3% by weight moisture measured on a moisture balance).

For example, the heating may occur at a temperature having an intake heat of from 110-130° C. and an exhaust heat of from 65-85° C. (e.g., from 70-78° C.). In some embodiments, the second liquid composition comprises the active (e.g., nicotine) dissolved in a solvent (e.g., aqueous solvent optionally comprising a humectant) such as propylene glycol, glycerol, water, or a combination thereof. In various aspects, following said spraying (e.g., prior to said extruding), the sprayed mixture is further mixed (e.g., for a time period of from 10 seconds to 1 minute). In certain aspects, the extruding step is performed in an extruder (e.g., having a die having from 0.3-2 mm holes (e.g., from 0.5-1.5 mm, from 1-1.5 mm)). The extruder may be operated at from 10-1800 rpm (e.g., 10-600 rpm). In certain aspects, the method further comprises:
f1) screening the spheronized extrudate (e.g., by passing through a third mesh and being collected in a fourth mesh).

The disclosure also includes kit comprising a plurality of the pouch systems described herein. By leveraging different types of particles in the pouch, different types of products can be mixed in the same container. In some embodiments, the kit may comprise a container and multiple pouch systems contained therein (e.g., from 2 to 10, 2 to 50, 2 to 10), and at least two of the multiple pouch systems have different characteristics. For example, at least two of the multiple pouch systems may have different flavors and/or different colorants and/or different release profiles and/or different sensorial properties. For example, the kit may comprise a container (e.g., a can) which may contain a first and second pouch system, wherein the first and second systems have different flavors and the first and second system each comprise a plurality of particles having different colorants. In some embodiments, the first system may comprise a plurality of red colored particles (e.g., mixed with white particles) and a portion of all the particles in the first system is strawberry flavored; and the second system may comprise a plurality of yellow-colored particles (e.g., mixed with white particles) and a portion of the particles in the second system is lemon flavored.

### BRIEF DESCRIPTION OF FIGURES

FIG. 1 provides dissolution data for pouches having spheronized extrudates of the present disclosure (Batch 1 which does not contain a low-viscosity polymer, Batch 2 which does not contain a high-viscosity polymer, and Batch 3 which contains a combination of a low-viscosity polymer and a high-viscosity polymer).

FIG. 2 provides dissolution data for several systems of the present disclosure as compared to a 6 mg ZYN® product.

FIG. 3 provides dissolution data for several systems of the present disclosure as compared to a 6 mg ZYN® product.

FIG. 4 provides dissolution data for several systems of the present disclosure as compared to a 3 mg ZYN® product.

FIG. 5 provides dissolution data for systems of the present disclosure affording comparison of release profiles as a function of active concentration.

FIG. 6 provides dissolution data for a system of the present disclosure as compared to a 6 mg ZYN® product.

### DETAILED DESCRIPTION

Detailed embodiments of the present disclosure are disclosed herein; however, it is to be understood that the disclosed embodiments are merely illustrative of the disclosure that may be embodied in various forms. In addition, each of the examples given in connection with the various embodiments of the disclosure is intended to be illustrative, and not restrictive.

All terms used herein are intended to have their ordinary meaning in the art unless otherwise provided. All concentrations are in terms of percentage by weight of the specified component relative to the entire weight of the topical composition, unless otherwise defined. Reference to a “first” or “second” component does not indicate the “first” component or “second” component are different unless indicated otherwise.

As used herein, “a” or “an” shall mean one or more. As used herein when used in conjunction with the word “comprising,” the words “a” or “an” mean one or more than one. As used herein “another” means at least a second or more.

As used herein, all ranges of numeric values include the endpoints and all possible values disclosed between the disclosed values. The exact values of all half-integral numeric values are also contemplated as specifically disclosed and as limits for all subsets of the disclosed range. For example, a range of from 0.1% to 3% specifically discloses a percentage of 0.1%, 1%, 1.5%, 2.0%, 2.5%, and 3%. Additionally, a range of 0.1 to 3% includes subsets of the original range including from 0.5% to 2.5%, from 1% to 3%, from 0.1% to 2.5%, etc. It will be understood that the sum of all weight % of individual components will not exceed 100%. Numerical values provided will be understood into include a degree of variance from the numerical value that one of ordinary skill in the art would understand is within acceptable tolerances for the particular parameter such as within a range of 90% to 110% of the indicated value, unless otherwise indicated. All numerical values may include the exact value provided.

By “consist essentially” it is meant that the ingredients include only the listed components along with the normal impurities present in commercial materials and with any other additives present at levels which do not affect the operation of the embodiments disclosed herein, for instance at levels less than 5% by weight or less than 1% or even 0.5% by weight.

The identification of a particular agent as having a certain activity or classification is not limiting, unless otherwise indicated, and does not preclude the same agent from having additional activities or classifications.

Systems are provided comprising:
a) a pouch; and
b) a plurality of particles comprising an active and contained within the pouch;
wherein, at least a portion of the particles optionally comprise a binder and/or a colorant and/or a sweetener and/or a flavorant and/or a high-viscosity polymer and/or a low-viscosity polymer and/or a buffer.

The particle size of any plurality of particles may refer to the average particle size as determined according to European Pharmacopoeia 9.1 when using test method 2.9.38 particle size distribution estimation by analytical sieving. In some embodiments, the average particle size in the plurality of particles (or the first plurality of particles or the second plurality of particles) may be independently less than ratio

(e.g., less than 1 mm, less than 500 μm, less than 200 μm). The average particle size may be determined by, for example, dynamic light scattering.

Suitable binders or fillers include Gum Arabic, Methyl Cellulose, Liquid glucose, Tragacanth, Ethyl Cellulose, Gelatin, Hydroxy Propyl Methyl Cellulose (HPMC), Starches (natural or modified), Hydroxy Propyl Cellulose (HPC), Pregelatinized Starch, Sodium Carboxy Methyl Cellulose (NaCMC), Alginic Acid, Polyvinyl Pyrrolidone (PVP), Maltodextrin (MD); Cellulose, Polyethylene Glycol (PEG), Polyvinyl Alcohols, Polymethacrylates, Copovidone or Microcrystalline Cellulose (MCC), alone or in combination. According to an embodiment of the invention, the one or more binders comprises one or more cellulose binders. In an embodiment of the invention the one or more binders comprises microcrystalline cellulose (MCC), hydroxypropyl cellulose (HPC) or hydroxypropylmethyl cellulose (HPMC) or any combination thereof.

In some embodiments, the filler is a cellulose material or a cellulose derivative. One particularly suitable filler for use in the beads described herein is microcrystalline cellulose (“MCC”). The MCC may be synthetic or semi-synthetic, or it may be obtained entirely from natural celluloses. The mcc may be selected from the group consisting of AVICEL® grades PH-100, PH-102, PH-103, PH-105, PH-112, PH-113, PH-200, PH-300, PH-302, VIVACEL® grades 101, 102, 12, 20 and EMOCEL® grades 50M and 90M, and the like, and mixtures thereof.

The polymers of the present disclosure, including the high-viscosity or low-viscosity polymer may be water-soluble synthetic or semi-synthetic non-ionic polymer, or both. In some embodiments, the polymer may be a starch and, particularly, a modified starch having suitable viscosity characteristics as described herein.

The starch as used herein may refer to pure starch from any source, modified starch, or starch derivatives. Starch is present, typically in granular form, in almost all green plants and in various types of plant tissues and organs (e.g., seeds, leaves, rhizomes, roots, tubers, shoots, fruits, grains, and stems). Starch can vary in composition, as well as in granular shape and size. Starches derived from various sources can be used. For example, major sources of starch include cereal grains (e.g., rice, wheat, and maize) and root vegetables (e.g., potatoes and cassava). Other examples of sources of starch include acorns, arrowroot, arracacha, bananas, barley, beans (e.g., favas, lentils, mung beans, peas, chickpeas), breadfruit, buckwheat, canna, chestnuts, colacasia, katakuri, kudzu, malanga, millet, oats, oca, Polynesian arrowroot, sago, sorghum, sweet potato, quinoa, rye, tapioca, taro, tobacco, water chestnuts, and yams.

Certain starches used in the particles (particularly those being used as the high-viscosity or low viscosity polymer) are modified starches. Some starches have been developed by genetic modifications, and are considered to be modified starches. Other starches are obtained and subsequently modified. For example, modified starches can be starches that have been subjected to chemical reactions, such as esterification, etherification, oxidation, depolymerization (thinning) by acid catalysis or oxidation in the presence of base, bleaching, transglycosylation and depolymerization (e.g., dextrinization in the presence of a catalyst), cross-linking, enzyme treatment, acetylation, hydroxypropylation, and/or partial hydrolysis. Other starches are modified by heat treatments, such as pregelatinization, dextrinization, and/or cold water swelling processes. Among the contemplated modified starches are the succinates, alkenyl succinates, diethylaminoethyl ethers, phthalates, sulfonates, carboxymethylated and chlorinated derivatives of native starches or thermally converted native starches. For example, the modified starches used may be the alkenyl succinates, and succinates of waxy maize starch. Certain modified starches include monostarch phosphate, distarch glycerol, distarch phosphate esterified with sodium trimetaphosphate, phosphate distarch phosphate, acetylated distarch phosphate, starch acetate esterified with acetic anhydride, starch acetate esterified with vinyl acetate, acetylated distarch adipate, acetylated distarch glycerol, hydroxypropyl starch, hydroxypropyl distarch glycerol, and starch sodium octenyl succinate. In various implementations, the modified starch is an octenyl succinic anhydride (OSA) starch, which may be a natural source treated with octenyl succinic anhydride or a salt thereof (e.g., sodium OSA). In these modified starches, the number of hydroxyl groups esterified with the OSA may be vary between 0.1% to 10% (e.g., 0.5% to 4%). Such modification may alter the properties of the starch (e.g., to create high viscosity polymers, to create low viscosity polymers). In particular embodiments, the modified starch is octenylbutanedioate amylodectrin.

In some embodiments, the first plurality of particles comprises a first core (e.g., comprising a filler or binder such as Gum Arabic, Methyl Cellulose, Liquid glucose, Tragacanth, Ethyl Cellulose, Gelatin, Hydroxy Propyl Methyl Cellulose (HPMC), Starches (natural or modified), Hydroxy Propyl Cellulose (HPC), Pregelatinized Starch, Sodium Carboxy Methyl Cellulose (NaCMC), Alginic Acid, Polyvinyl Pyrrolidone (PVP), Maltodextrin (MD); Cellulose, Polyethylene Glycol (PEG), Polyvinyl Alcohols, Polymethacrylates, Copovidone or Microcrystalline Cellulose (MCC), alone or in combination) and a first coating on the first core (e.g., a coating comprising a saliva-soluble binder such as hydroxypropyl methylcellulose (HPMC), hydroxypropyl cellulose (HPC), methyl cellulose (MC), polyvinylpyrrolidone and mixtures thereof). In some embodiments, the second plurality of particles comprises a second core (e.g., comprising a filler or binder such as Gum Arabic, Methyl Cellulose, Liquid glucose, Tragacanth, Ethyl Cellulose, Gelatin, Hydroxy Propyl Methyl Cellulose (HPMC), Starches (natural or modified), Hydroxy Propyl Cellulose (HPC), Pregelatinized Starch, Sodium Carboxy Methyl Cellulose (NaCMC), Alginic Acid, Polyvinyl Pyrrolidone (PVP), Maltodextrin (MD); Cellulose, Polyethylene Glycol (PEG), Polyvinyl Alcohols, Polymethacrylates, Copovidone or Microcrystalline Cellulose (MCC), alone or in combination) and a second coating on the second core (e.g., a coating comprising a saliva-soluble binder such as hydroxypropyl methylcellulose (HPMC), hydroxypropyl cellulose (HPC), methyl cellulose (MC), polyvinylpyrrolidone and mixtures thereof). The first and/or second coating may have a uniform thickness. In some embodiments, the thickness of the first and/or second coating may be from 10 μm to 100 μm. In some embodiments, the first and/or second coating may contain an additional agent, such as a pH adjusting agent or an active agent (e.g., nicotine or pharmaceutically acceptable salts and/or cyclodextrin forms thereof, polacrilex, IMOTINE® (6-methylnicotine) or pharmaceutically acceptable salts and/or cyclodextrin forms thereof, caffeine or pharmaceutically acceptable salts thereof, lidocaine, synephrine, kratom such as Mitragyna speciosa extracts or any alkaloids present therein such as mitragynine). In some embodiments, the first and/or second core may contain an additional agent, such as a pH adjusting agent or an active agent (e.g., nicotine or pharmaceutically acceptable salts and/or cyclodextrin forms thereof, polacrilex, IMOTINE® (6-methylnicotine) or pharmaceutically acceptable salts and/or cyclodextrin forms thereof, caffeine or pharmaceutically acceptable salts thereof, lidocaine, synephrine, kratom such as Mitragyna speciosa extracts or any alkaloids present therein such as mitragynine). In some embodiments, the first and second core are the same (e.g., formed from the same extrudate).

Leveraging the properties of the presently disclosed particle systems, the release profiles of different actives and/or flavoring agents can be controlled to provide unique active and flavor delivery pouches. For example, the pouch may comprise actives (different actives, the same active) located in the different portions of the particles and/or may result in different release profiles. Any active and/or flavorant may independently have immediate release, extended release, and/or delayed release from the particles (e.g., from the core, from the coating, from the first core, from the first coating, from the second core, from the second coating) from any plurality of particles described herein. In some embodiments, the first coating and the first core contain different active ingredients (e.g., active ingredients independently selected from absent, nicotine or pharmaceutically acceptable salts and/or cyclodextrin forms thereof, polacrilex, IMOTINE® (6-methylnicotine) or pharmaceutically acceptable salts and/or cyclodextrin forms thereof, caffeine or pharmaceutically acceptable salts thereof, lidocaine, synephrine, kratom such as Mitragyna speciosa extracts or any alkaloids present therein such as mitragynine, wherein at least one of the first coating and the first core contain an active ingredient). In certain aspects, the first coating and the second coating contain different active ingredients. In some embodiments, the first coating and the second core contain different active ingredients. In some embodiments, the second coating and the second core contain different active ingredients. In certain aspects, the first core and the second core contain different active ingredients. In various implementations, the first coating and the first core contain the same active ingredient (e.g., with different release profiles). In certain aspects, the first coating and the second coating contain the same active ingredients. In some embodiments, the first coating and the second core contain the same active ingredients. In some embodiments, the second coating and the second core contain the same active ingredients. In some embodiments, the first core and the second core contain the same active ingredients.

In some embodiments, the first coating and the first core contain different flavorants. In certain aspects, the first coating and the second coating contain different flavorants. In some embodiments, the first coating and the second core contain different flavorants. In some embodiments, the second coating and the second core contain different flavorants. In certain aspects, the first core and the second core contain different flavorants. In various implementations, the first coating and the first core contain the same flavorant. In certain aspects, the first coating and the second coating contain the same flavorants. In some embodiments, the first coating and the second core contain the same flavorants. In some embodiments, the second coating and the second core contain the same flavorants. In some embodiments, the first core and the second core contain the same flavorants. In some embodiments, the first core and the second core are formed from the same extrudate and the first and second coatings are optionally different.

In some embodiments, the different plurality of particles may have one or more (e.g., only one, only two, only three) O characteristics different between them. For example, in some embodiments, the first plurality and the second plurality particles differ only by the presence of a colorant (e.g., in the core, in a coating). In some embodiments spec, the first and second plurality of particles have similar densities (e.g., within 50% of one another). In some embodiments, the first and second pluralities of particles have a particle size difference of within 100% of one another. In some embodiments, the plurality of particles comprises multiple pluralities of particles (e.g., from 1-5 particles each with independently at least one distinct characteristic). All differences associated with that characteristic difference may be included in the identification of a characteristic difference. For example, a first plurality of particles may be formed from a first extrudate made from a composition (e.g., sprayed mixture) comprising a binder, low viscosity polymer, and active, and the second plurality of particles may be formed from the a second extrudate formed by addition of a colorant to the composition. This may affect various properties including weight ratios, densities, particle sizes, but may be considered a single characteristic difference (e.g., colorant) between the pluralities of particles.

In some embodiments, the one or more buffering agents is selected from the group consisting of tri(hydroxymethyl)aminomethane buffering agents, phosphate buffering agents, carbonate buffering agents, and combinations thereof. In some embodiments, the buffering agent is present in an amount of less than (or from 1% to) 30% (e.g., 5%-25%, 10%-20%) by weight of the composition. The buffer may comprise, for example, disodium phosphate, sodium carbonate, sodium citrate, sodium bicarbonate, or a combination thereof.

In some embodiments, the composition comprises one or more sugar alcohols such as sorbitol, erythritol, xylitol, lactitol, maltitol, mannitol, isomalt, and combinations thereof. In some embodiments, the composition further comprises one or more high-intensity sweeteners. In some embodiments, the composition further comprises fillers, such as calcium carbonate and/or talc and/or cellulose fibers and/or microcrystalline cellulose.

The active substance formulated in the plurality of particles (e.g., the first plurality of particles, the second plurality of particles) may for example be selected from nutraceuticals, nootropics and psychoactives. The active substance, which may be independently selected in any plurality of particles, may be naturally occurring or synthetically obtained. The active substance may comprise for example nicotine or pharmaceutically acceptable salts or cyclodextrins thereof, IMOTINE® (6-methylnicotine) or pharmaceutically acceptable salts or cyclodextrins thereof, caffeine, kratom such as Mitragyna speciosa extracts or any alkaloids present therein such as mitragynine, mytragynine, synephrine, lidocaine, taurine, theine, vitamins such as B6 or B12 or C, melatonin, cannabinoids, or constituents, derivatives, or combinations thereof. The active substance may comprise one or more constituents, derivatives or extracts of tobacco, cannabis or another botanical. In some embodiments, the active substance comprises nicotine. In some embodiments, the active substance comprises caffeine, melatonin or vitamin B12. Caffeine may include synthetic caffeine and/or natural caffeine, such as coffee-bean-extracted caffeine.

In some embodiments, the particles comprise a nicotine source which may be a substance that contains nicotine and has a physiological effect on the human body for the benefit of the human body or part thereof. Nicotine sources include tobacco, free nicotine base, salts of nicotine, or nicotine ion-exchange resins.

The nicotine may be any form, including free base nicotine, nicotine salts, nicotine bound to ion exchange resins, nicotine bound to zeolites; nicotine bound to cellulose, such as microcrystalline cellulose, such as of microbial origin, or starch microspheres, nicotine bound to CaCO₃, and mixtures thereof. Thus, when referring to nicotine amounts, the amount refers to the amount of pure nicotine. It will be understood that the nicotine described in the Examples, unless otherwise indicated, is free base nicotine.

Salts of any active, such a nicotine salt, are generally an ionized form of an active bonded electrostatically to a counterion. For example, the particles may comprise a nicotine salt selected from nicotine ascorbate, nicotine aspartate, nicotine benzoate, nicotine monotartrate, nicotine bitartrate, nicotine chloride (e.g., nicotine hydrochloride and nicotine dihydrochloride), nicotine citrate, nicotine fumarate, nicotine gensitate, nicotine lactate, nicotine mucate, nicotine laurate, nicotine levulinate, nicotine malate nicotine perchlorate, nicotine pyruvate, nicotine salicylate, nicotine sorbate, nicotine succinate, nicotine zinc chloride, nicotine sulfate, nicotine tosylate and hydrates thereof (e.g., nicotine zinc chloride monohydrate).

The active compound (e.g., nicotine) may be incorporated as a complex with a cyclodextrin. Without wishing to be bound by any theory, cyclodextrins form inclusion complexes with an active compound through a process in which the water molecules located in the central cavity are replaced by either the whole active compound molecule, or by some lipophilic portion of the active compound structure. The tridimensional structure of the cyclodextrin molecule may provide a hydrophobic barrel that can bind and protect the active compound. Once included in the cyclodextrin cavity (i.e., the hydrophobic barrel), the drug molecules may be dissociated through complex dilution by replacement of the included drug by some other suitable molecule, and the drug may be transferred to the matrix for which it has the highest affinity. Since no covalent bonds are typically formed or broken during the drug cyclodextrin complex formation, the complexes may be in dynamic equilibrium with free drug and cyclodextrin molecules (R. A. Rajewski, J. Pharm. Sci. 85.11, 1142-1169 (1996), which is hereby incorporated by reference in its entirety). The cyclodextrin may be an α-cyclodextrin, β-cyclodextrin, or γ-cyclodextrin.

Contacting the active compound with at least one cyclodextrin may include dissolving or suspending cyclodextrin in a solvent or mixture of solvents to form a first solution or suspension. Similarly, the active compound may be dissolved or suspended in the same or different solvent or mixture of solvents to form a second solution or suspension. The first solution or suspension may then be combined to form the present complex between active compound and the at least one cyclodextrin. The complex may then be separated from the solution and optionally purified, resulting in a complex of stabilized active.

Contacting an active compound with at least one cyclodextrin may alternatively include dissolving or suspending at least one cyclodextrin in a solvent or mixture of solvents to form a solution or suspension, and then adding an active compound to the solution or suspension to form the present complex. Contacting an active compound with at least one cyclodextrin may be conducted by other methods. For example, a solvent may be utilized which will fully dissolve both the active compound and the cyclodextrin. In another embodiment, the cyclodextrin may be dissolved or suspended in a solvent or mixture of solvents and then placed on a rotovaporator. The active compound may then be sprayed directly into the solution or suspension, either as a neat form or as a solution or suspension of active compound in a solvent or mixture of solvents. The contacting may also be accomplished by use of a biphasic solvent system. For example, the active compound may be combined in separate, immiscible solvents (either as suspensions or in solution). The immiscible solvents may then be thoroughly mixed until a complex is formed. The complex may then be isolated via one of the isolation techniques discussed herein. It may be desirable to conduct the contact step in the absence of solvents. For example, in a spray drying technique, a mist of nitroalkene may be sprayed or misted on neat cyclodextrin to produce the present complex.

The particles may comprise sweeteners, such as bulk sweeteners, sugar sweeteners, sugar substitute sweeteners, artificial sweeteners, high-intensity sweeteners, or any combination thereof.

Useful sugar sweeteners are saccharide-containing components include sucrose, dextrose, maltose, dextrins, trehalose, D-tagatose, dried invert sugar, fructose, levulose, galactose, corn syrup solids, and the like, alone or in combination. Non-sugar sweeteners may include sorbitol, mannitol, xylitol, hydrogenated starch hydrolysates, maltitol, isomalt, erythritol, lactitol and the like, alone or in combination. High intensity artificial sweetening agents can also be used alone or in combination in the particles. For example, high intensity sweeteners include, but are not limited to sucralose, aspartame, salts of acesulfame, alitame, saccharin and its salts, cyclamic acid and its salts, glycyrrhizin, dihydrochalcones, thaumatin, monellin, stevioside (natural intensity sweetener) and the like, alone or in combination. In some embodiments, the sweetener comprises neotame. In order to provide longer lasting sweetness, it may be desirable to encapsulate or otherwise control the release of at least a portion of the artificial sweeteners. Techniques such as wet granulation, wax granulation, spray drying, spray chilling, fluid bed coating, conservation, encapsulation in yeast cells and fiber extrusion may be used to achieve desired release characteristics.

Non-limiting examples of suitable flavor-providing ingredients include natural flavors, artificial flavors, spices, seasonings, and the like. These include synthetic flavor oils and flavoring aromatics and/or oils, oleoresins, essences, extracts, concentrates, and distillates, and combinations thereof. Flavor oils include spearmint oil, cinnamon oil, oil of wintergreen (methyl salicylate), peppermint oil, Japanese mint oil, clove oil, bay oil, anise oil, eucalyptus oil, thyme oil, cedar leaf oil, oil of nutmeg, allspice, oil of sage, mace, oil of bitter almonds, and cassia oil, useful flavoring agents include artificial, natural and synthetic fruit flavors such as vanilla, and citrus oils including lemon, orange, lime, grapefruit, yuzu, sudachi, and fruit essences including apple, pear, peach, grape, raspberry, blackberry, gooseberry, blueberry, strawberry, cherry, plum, prune, raisin, cola, guarana, neroli, pineapple, apricot, banana, melon, apricot, cherry, tropical fruit, mango, mangosteen, pomegranate, papaya, and so forth. In some embodiments, the other flavorant ingredients include aldehydes and esters such as cinnamyl acetate, cinnamaldehyde, citral diethylacetal, dihydrocarvyl acetate, eugenyl 49 formate, p-methylamisol, and so forth can be used. Further examples of aldehyde flavorings include acetaldehyde (apple), benzaldehyde (cherry, almond), anisic aldehyde (licorice, anise), cinnamic aldehyde (cinnamon), citral, i.e., alpha-citral (lemon, lime), neral, i.e., beta-citral (lemon, lime), decanal (orange, lemon), ethyl vanillin (vanilla, cream), heliotrope, i.e., piperonal (vanilla, cream), vanillin (vanilla, cream), alpha-amyl cinnamaldehyde (spicy fruity flavors), butyraldehyde (butter, cheese), valeraldehyde (butter, cheese), citronellal (modifies, many types), decanal (citrus fruits), aldehyde C-8 (citrus fruits), aldehyde C-9 (citrus fruits), aldehyde C-12 (citrus fruits), 2-ethyl butyraldehyde (berry fruits), hexenal, i.e., trans-2 (berry fruits), tolyl aldehyde (cherry, almond), veratraldehyde (vanilla), 2,6-dimethyl-5-heptenal, i.e., melonal (melon), 2,6-dimethyloctanal (green fruit), and 2-dodecenal (citrus, mandarin), and the like. Some compositions of the present disclosure may include glucose syrup, and/or carriers such as gelatin, sugar, fructose, and corn syrup. Pectin from apple or citrus may be used. Several flavorings include citrus flavor, berry mix flavor, tropical fruit flavor, ginger flavor, tangerine flavor, and tart cherry. Typical colors include carrot juice color, beet juice color, genipap fruit color, spinach color, and artificial FD&C colors.

The particles of the present disclosure (e.g., first plurality of particles, second plurality of particles) may independently contain at least one of a colorant, opacifier, flavorant, sweetener, preservative (e.g., potassium sorbate, sodium benzoate, butylated hydroxytoluene (BHT)), antioxidant (e.g., citric acid, Vitamin E or a derivative thereof, a tocopherol, epicatechol, epigallocatechol, epicgallocatechol gallate, erythrorbic acid, sodium erythorbate, 4-hexylresorcinol, theaflavin, theaflavin monogallate A or B, theflavin digallate, phenolic acids, glycosides, quercetin, isoquercetin, hyperoside, polyphenols, catechols, reservatrols, olenropein, butylated hydroxyanisole (BHA), butylated hydroxytoluene (BHT), tertiary butylhydroquinone (TBHQ)), cooling agent (e.g., WS-3, WS-5, WS-12, WS-23, menthol), sour/tartening agents (e.g., citric acid, malic acid, ascorbic acid), spicy agents (e.g., capsaicin, allyl isothiocyanate, piperine), salty agents (e.g., sodium chloride, ammonium chloride), taste enhancers (e.g., mono-sodium glutamate, capaicin), emulsifier (e.g., lecithin, phospholipids), surfactants (e.g., polysorbate 80), or gellant. These components may be independently present in the core and/or coating. In some embodiments, the emulsifier is a liposomal emulsifier such as a phospholipid. Suitable colorants include, but are not limited to, azo dye, quinophthalone dye, triphenylmethane dye, xanthene dye, iron oxide, iron hydroxide, titanium dioxide, sunset yellow, allura red, amaranth, koki neil red, azogeranin, tartrazine, brilliant black, canthaxanthin, patent blue, fast green, brilliant blue, acid green, erythrosine, quinoline yellow, indigotin, curcumin, carbon black and/or combinations thereof. In some embodiments the particles, (e.g., the core, the coating) comprise a colorant that is a whitening agent such as titanium dioxide. Suitable flavorants include, but are not limited to, a natural flavor oil, an artificial flavor oil, a synthetic flavor oil, a flavoring aromatic, a flavoring oils, an oleoresin, plant extract, leaf extract, flower extract, fruit extract, citrus oil, spearmint oil, peppermint oil, Eucalyptus oil, nutmeg oil, allspice oil, mace, almond oil, menthol oil, citrus oil, lemon oil, orange oil, lime oil, grapefruit oil and/or combinations thereof. In embodiments, compositions may include citrus flavor, ginger flavor, berry flavor, cherry flavor, vanilla flavor or combinations thereof. Suitable sweeteners include, but are not limited to, agave syrup, Stevia, erythritol, xylitol, sorbitol, yacon syrup, aspartame, saccharin, cyclamate, sucralose, monk fruit extract and/or combinations thereof. In some embodiments, the sweetener can include a sugar alcohol such as erythritol, xylitol, sorbitol, or glycerin. In some embodiments, the sweetener can include a disaccharide such as sucrose, lactose, or maltose. In various implementations, the composition may include sodium chloride. The compositions may include monosaccharides, such as glucose, fructose, allulose or galactose; disaccharides such as sucrose, lactose or maltose; and/or sugar alcohols as sorbitol, mannitol, maltitol, xylitol, erythritol, or isomalt.

The particles may comprise, for example, one or more dyes, toners or lakes. Lakes generally refer to a colorant prepared from a water-soluble organic dye (e.g., D&C or FD&C) which has been precipitated onto an insoluble reactive or adsorptive substratum or diluent. In some embodiments, the organic pigments may be azo dye based or comprise one or more azo moieties. Typically, azo based pigments are organic compounds comprising the linkage-N═N—. The term “D&C” means drug and cosmetic colorants that are approved for use in drugs and cosmetics by the FDA. The term “FD&C” means food, drug, and cosmetic colorants which are approved for use in foods, drugs, and cosmetics by the FDA. Certified D&C and FD&C colorants are listed in 21 C.F.R. § 74.101 et seq. and include the FD&C colors Blue 1, Blue 2, Green 3, Orange B, Citrus Red 2, Red 3, Red 4, Red 40, Yellow 5, Yellow 6, Blue 1, Blue 2; Orange B, Citrus Red 2; and the D&C colors Blue 4, Blue 9, Green 5, Green 6, Green 8, Orange 4, Orange 5, Orange 10, Orange 11, Red 6, Red 7, Red 17, Red 21, Red 22, Red 27, Red 28, Red 30, Red 31, Red 33, Red 34, Red 36, Red 39, Violet 2, Yellow 7, Yellow 8, Yellow 10, Yellow 11, Blue 4, Blue 6, Green 5, Green 6, Green 8, Orange 4, Orange 5, Orange 10, Orange 11, and so on. For example, the pigmented composition may comprise D&C Red 19 (e.g., CI 45170, CI 73360 or CI 45430); D&C Red 9 (CI 15585); D&C Red 21 (CI 45380); D&C Orange 4 (CI 15510); D&C Orange 5 (CI 45370); D&C Red 27 (CI 45410); D&C Red 13 (CI 15630); D&C Red 7 (CI 15850:1); D&C Red 6 (CI 15850:2); D&C Yellow 5 (CI 19140); D&C Red 36 (CI 12085); D&C Orange 10 (CI 45475); D&C Yellow 19 (CI 15985); FD&C Red 40 (CI 16035); FD&C Blue 1 (CI 42090); FD&C Yellow 5 (CI 19140); or any combinations thereof. In certain implementations, the composition may comprise one or more azo based organic pigments.

Substrates suitable for forming lakes include, without limitation, mica, bismuth oxychloride, sericite, alumina, aluminum, copper, bronze, silver, calcium, zirconium, barium, and strontium, titanated mica, fumed silica, spherical silica, polymethylmethacrylate (PMMA), micronized TEFLON, boron nitride, acrylate copolymers, aluminum silicate, aluminum starch octenylsuccinate, bentonite, calcium silicate, cellulose, chalk, corn starch, diatomaceous earth, fuller's earth, glyceryl starch, hectorite, hydrated silica, kaolin, magnesium aluminum silicate, magnesium trisilicate, maltodextrin, montmorillonite, microcrystalline cellulose, rice starch, silica, talc, mica, titanium dioxide, zinc laurate, zinc myristate, zinc rosinate, alumina, attapulgite, calcium carbonate, calcium silicate, dextran, nylon, silica silylate, silk powder, sericite, soy flour, tin oxide, titanium hydroxide, trimagnesium phosphate, walnut shell powder, and mixtures thereof.

Suitable lakes include, without limitation, those of red dyes from the monoazo, disazo, fluoran, xanthene, or indigoid families, such as Red 4, 6, 7, 17, 21, 22, 27, 28, 30, 31, 33, 34, 36, and Red 40; lakes of yellow pyrazole, monoazo, fluoran, xanthene, quinoline, dyes or salt thereof, such as Yellow 5, 6, 7, 8, 10, and 11; lakes of violet dyes including those from the anthroquinone family, such as Violet 2 as well as lakes of orange dyes, including Orange 4, 5, 10, 11, and the like.

In embodiments of the present invention, the particles comprise humectants, such as propylene glycol or glycerol. In some embodiments, the particles (or a plurality of particles such as the first plurality of particles or the second plurality of particles) comprise less than 5% or less than 1% or no humectants by weight.

In various implementations, any component may be derived from plant material. For example, the binder may comprise plant matter (e.g., plant material in its natural form or material derived from natural plant materials such as extracts or isolates from plant materials or treated plant materials (e.g., plant material subjected to heat treatment, fermentation, chemical reaction such as esterification, etherification, oxidation, depolymerization, by acid catalysis, oxidation in the presence of base, bleaching, transglycosylation, depolymerization, cross-linking, enzyme treatment, acetylation, hydroxy-alkylation such as hydroxypropylation or hydroxypropylmethylation, hydrolysis including partial hydrolysis, pre-gelatinization, dextrinization, cold water swelling) in the particles. In various implementations, the plant matter is not bleached. In certain embodiments, a portion or all components of the particles (e.g., plant matter, binder, low viscosity polymer, high viscosity polymer, filler, active) is unbleached.

For example, in some embodiments, the plurality of particles comprises solvent (e.g., water, ethanol, a combination thereof), a sweetener (e.g., sucralose), a taste enhancer (e.g., monosodium glutamate), an active inclusion complex (e.g., nicotine/β-cyclodextrin), a preservative (e.g., butylated hydroxytoluene), filler (e.g., talc), a sweetener (e.g., maltodextrin, mannitol, sucralose, isomalt, combination thereof), binder (e.g., microcrystalline cellulsoe) low-viscosity polymer modified (e.g., starch such as OSA-starch, hydroxypropylmethylcellulose, combination thereof), buffer (e.g., trisodium citrate, disodium citrate, sodium bicarbonate, combination thereof), sour/tartening agents (e.g., ascorbic acid), and pigments. In various implementations the plurality of particles further comprise one or more cooling agents and/or spicy agents and/or flavorants such as flavoring aromatics. Any or all of these components may be used to also make the first and/or second plurality of particles (particularly the cores of these particles). In particular embodiments, the first plurality of particles and the second plurality of particles comprise the same low viscosity polymer (e.g., in a weight percentage of low viscosity polymer in each plurality of particles of within 10% of one another).

The films of the present disclosure may include water-soluble polymers such as polyvinyl alcohol (PVOH) polymers. Other water soluble polymers for use in addition to the PVOH polymers and PVOH copolymers in the blend can include, but are not limited to modified polyvinyl alcohols, polyacrylates, water-soluble acrylate copolymers, polyvinyl pyrrolidone, polyethyleneimine, pullulan, water-soluble natural polymers including, but not limited to, guar gum, gum Acacia, xanthan gum, carrageenan, and starch, water-soluble polymer derivatives including, but not limited to, modified starches, ethoxylated starch, and hydroxypropylated starch, copolymers of the forgoing and combinations of any of the foregoing. Yet other water-soluble polymers can include polyalkylene oxides, polyacrylamides, polyacrylic acids and salts thereof, celluloses, cellulose ethers, cellulose esters, cellulose amides, polyvinyl acetates, polycarboxylic acids and salts thereof, polyaminoacids, polyamides, gelatines, methylcelluloses, carboxymethylcelluloses and salts thereof, dextrins, ethylcelluloses, hydroxyethyl celluloses, hydroxypropyl methylcelluloses, maltodextrins, and polymethacrylates. Such water-soluble polymers, whether PVOH or otherwise are commercially available from a variety of sources. Any of the foregoing water-soluble polymers are generally suitable for use as film-forming polymers. In general, the water-soluble film can include copolymers and/or blends of the foregoing resins.

The coatings may include one or more water soluble polymers (including, but not limited to PVOH polymers and PVOH copolymers) can be characterized by a viscosity in a range of 3.0 to 27.0 cP, 4.0 to 24.0 cP, 4.0 to 23.0 cP, 4.0 cP to 15 cP, or 6.0 to 10.0 cP, for example. The viscosity of a polymer is determined by measuring a freshly made solution using a Brookfield LV type viscometer with UL adapter as described in British Standard EN ISO 15023-2:2006 Annex E Brookfield Test method. It is international practice to state the viscosity of 4% aqueous polyvinyl alcohol solutions at 20° C. Polymeric viscosities specified herein in cP should be understood to refer to the viscosity of a 4% aqueous water-soluble polymer solution at 20° C., unless specified otherwise.

The viscosity of a water-soluble polymer (PVOH or otherwise) is correlated with the weight-average molecular weight (Mw) of the same polymer, and often the viscosity is used as a proxy for Mw. Thus, the weight-average molecular weight of the water-soluble polymers, including the first PVOH copolymer and second PVOH polymer, can be in a range of 30,000 to 175,000, or 30,000 to 100,000, or 55,000 to 85,000, for example.

The water-soluble film can contain other auxiliary agents and processing agents, such as, but not limited to, plasticizers, plasticizer compatibilizers, surfactants, lubricants, release agents, fillers, extenders, cross-linking agents, antiblocking agents, antioxidants, detackifying agents, antifoams, nanoparticles such as layered silicate-type nanoclays (e.g., sodium montmorillonite), whitening agents (e.g., titanium dioxide), aversive agents such as bitterants (e.g., denatonium salts such as denatonium benzoate, denatonium saccharide, and denatonium chloride; sucrose octaacetate; quinine; flavonoids such as quercetin and naringen; and quassinoids such as quassin and brucine) and pungents (e.g., capsaicin, piperine, allyl isothiocyanate, and resinferatoxin), and other functional ingredients, in amounts suitable for their intended purposes. Films that include plasticizers are beneficial. The amount of such agents can be up to 50 wt. %, 20 wt %, 15 wt %, 10 wt %, 5 wt. %, 4 wt % and/or at least 0.01 wt. %, 0.1 wt %, 1 wt %, or 5 wt %, individually or collectively. Preferably the total amount of such auxiliary agents and processing aids in the film is from 20% to 50%, more preferably from 25% to 40%.

The plasticizer can include, but is not limited to, glycerin, diglycerin, sorbitol, ethylene glycol, diethylene glycol, triethylene glycol, dipropylene glycol, tetraethylene glycol, propylene glycol, polyethylene glycols up to 400 MW, neopentyl glycol, trimethylolpropane, polyether polyols, sorbitol, 2-methyl-1,3-propanediol, ethanolamines, and a mixture thereof. A preferred plasticizer is glycerin, sorbitol, triethyleneglycol, propylene glycol, diproyplene glycol, 2-methyl-1,3-propanediol, trimethylolpropane, or a combination thereof. The total amount of the plasticizer can be in a range of 1 wt. % to 40 wt. %, or 10 wt. % to 40 wt. %, or 15 wt. % to 35 wt. %, or 20 wt. % to 30 wt. %, for example 25 wt. %, based on total film weight. Combinations of glycerin, dipropylene glycol, and sorbitol can be used. Alternatively, combinations of glycerin, trimethylolpropane and sorbitol can be used. Optionally, glycerin can be used in an amount of 5 wt % to 30 wt %, or 5 wt % to 20 wt %, e.g., 13 wt %. Optionally, dipropylene glycol or trimethylolpropane can be used in an amount of 1 wt. % to 20 wt. %, or 3 wt. % to 10 wt. %, for example 6 wt. %. Optionally, sorbitol can be used in an amount of 1 wt % to 20 wt %, or 2 wt % to 10 wt %, e.g., 5 wt %. The specific amounts of plasticizers can be selected in a particular embodiment based on desired film flexibility and processability features of the water-soluble coating. At low plasticizer levels, films may become brittle, difficult to process, or prone to breaking. At elevated plasticizer levels, films may be too soft, weak, or difficult to process for a desired use.

The systems may impart an increase or decreased sensation as compared to pouches having a different release profile (e.g., pouches having a slower release profile in the first five minutes). Additionally, the systems may impart an increased or decreased sensation by having at least two pluralities of particles with different properties (e.g., different color, different actives, different taste). For example, in some embodiments, the plurality of particles may comprise a colorant which, may aid in identifying the content (e.g., flavor), or provide an enhanced flavor profile based on a user's association with the color. The increase or decrease for any sensorial result each may be observed based on a difference of at least one point (e.g., one point, two points, three points, four points, five points, six points) on a 9-point scale for the sensorial result as evaluated by three or more reviewers (e.g., from 3 reviewers, 4 reviewers, 5 reviewers, 6 reviewers, 7 reviewers, 8 reviewers, 9 reviewers, 10 reviewers, 11 reviewers, 12 reviewers, 13 reviewers, 14 reviewers, 15 reviewers, 16 reviewers, 17 reviewers, 18 reviewers, 19 reviewers, 20 reviewers). Reviewers may be asked to ascertain the sensorial result assigning a numeric value on a scale from 1-9. The difference in composition is typically statistically significant having a p-value of less than 0.05. Rating these descriptors on a 9-point scale may provide a characterization of the compositions of the present disclosure.

The components in any of the particles (e.g., first plurality of particles, second plurality of particles) of the present disclosure such as the active agent or flavorant may have different release profiles. For example, in some embodiments the first plurality of particles has an immediate release, delayed release, or extended release of active and/or flavorant and the second plurality of particles has an immediate release, delayed release, or extended release of active and/or flavorant, wherein the first and second plurality of particles have different release profiles of active and/or flavorant. Components with a delayed release profile may indicate that 90% or more of the indicated component (e.g., active, flavorant) administered and contained within the particles, for example from within the core and/or coating, is delayed following administration (e.g., for more than 5 minutes or more than 10 minutes or more than 20 minutes or from 5 to 20 minutes), after oral administration, from being released from the particles. Extended release components may have 90% or more of the component (e.g., active, flavorant) contained within the particles, is released over a linear rate or nearly linear rate (e.g., release occurs with an R-squared value of greater than 0.9 or 0.95 for concentration released as a function of time) following administration. Immediate release components may release at 90% or more of the active therapeutic agent administered and contained within the formulation, for example, within the core and/or coating, is released within two hours or one hour or 30 minutes or 20 minutes or 10 minutes or 5 minutes following administration. Release profiles may be determined, for example, using USP Apparatus 2 Paddle operating at 40 RPM at 37° C.

In various implementations, the first plurality of particles comprises an active such as nicotine or pharmaceutically acceptable forms thereof (e.g., in the core, in the coating) and the second plurality of particles comprises an active such as caffeine or pharmaceutically acceptable forms thereof (e.g., in the core, in the coating). For example, the first plurality of particles may comprise nicotine (e.g., in the core, in the coating) with an immediate release dosage and the second plurality of particles comprises caffeine or pharmaceutically acceptable forms thereof (e.g., in the core, in the coating) with a sustained release profile. In some embodiments, the first plurality of particles comprises nicotine or pharmaceutically acceptable forms thereof (e.g., in the core, in the coating) with sustained release dosage and the second plurality of particles comprises caffeine or pharmaceutically acceptable forms thereof (e.g., in the core, in the coating) with an immediate release profile. In some embodiments, the first plurality of particles comprises nicotine or pharmaceutically acceptable forms thereof (e.g., in the core, in the coating) with extended release dosage and the second plurality of particles comprises caffeine or pharmaceutically acceptable forms thereof (e.g., in the core, in the coating) with an immediate release profile. In some embodiments, the first plurality of particles comprises nicotine or pharmaceutically acceptable forms thereof (e.g., in the core, in the coating) with immediate release dosage and the second plurality of particles comprises caffeine or pharmaceutically acceptable forms thereof (e.g., in the core, in the coating) with an extended release profile. In some embodiments, the first plurality of particles comprises nicotine or pharmaceutically acceptable forms thereof (e.g., in the core, in the coating) with sustained release dosage and the second plurality of particles comprises caffeine or pharmaceutically acceptable forms thereof (e.g., in the core, in the coating) with an immediate release profile. In some embodiments, the first plurality of particles comprises nicotine or pharmaceutically acceptable forms thereof (e.g., in the core, in the coating) with immediate release dosage and the second plurality of particles comprises caffeine or pharmaceutically acceptable forms thereof (e.g., in the core, in the coating) with a sustained release profile. In certain aspects, the first plurality of particles may comprise IMOTINE® or pharmaceutically acceptable forms thereof (e.g., in the core, in the coating) with an immediate release dosage and the second plurality of particles comprises caffeine or pharmaceutically acceptable forms thereof (e.g., in the core, in the coating) with a sustained release profile. In some embodiments, the first plurality of particles comprises IMOTINER or pharmaceutically acceptable forms thereof (e.g., in the core, in the coating) with sustained release dosage and the second plurality of particles comprises caffeine or pharmaceutically acceptable forms thereof (e.g., in the core, in the coating) with an immediate release profile. In some embodiments, the first plurality of particles comprises IMOTINE® or pharmaceutically acceptable forms thereof (e.g., in the core, in the coating) with extended release dosage and the second plurality of particles comprises caffeine or pharmaceutically acceptable forms thereof (e.g., in the core, in the coating) with an immediate release profile. In some embodiments, the first plurality of particles comprises IMOTINER or pharmaceutically acceptable forms thereof (e.g., in the core, in the coating) with immediate release dosage and the second plurality of particles comprises caffeine or pharmaceutically acceptable forms thereof (e.g., in the core, in the coating) with an extended release profile. In some embodiments, the first plurality of particles comprises IMOTINE® or pharmaceutically acceptable forms thereof (e.g., in the core, in the coating) with sustained release dosage and the second plurality of particles comprises caffeine or pharmaceutically acceptable forms thereof (e.g., in the core, in the coating) with an immediate release profile. In some embodiments, the first plurality of particles comprises IMOTINER or pharmaceutically acceptable forms thereof (e.g., in the core, in the coating) with immediate release dosage and the second plurality of particles comprises caffeine or pharmaceutically acceptable forms thereof (e.g., in the core, in the coating) with an sustained release profile. In various implementations, any active (e.g., the active in the first plurality of particles, the active in the second plurality of particles) may be part of an inclusion complex such as complexed with cyclodextrin (e.g., β-cyclodextrin/nicotine).

The pouches are typically containers formed by a web of a fibrous material enclosing a cavity. The pouch is designed for administration of an active ingredient in the oral cavity, and thus it is typically adapted for oral use, it is non-toxic and not water-soluble. The fibrous material may e.g. form a woven or non-woven web or fabric. The pouch may for example be sealed by bonding two corresponding pieces of web or fabric to each other along their edges to form a cavity for the oral composition. In order to release the active ingredients, the pouch is made water-permeable so as to allow saliva from the oral cavity to penetrate the pouch and enter the cavity, where the saliva can come into contact with the one or more active ingredients, whereby the one or more active ingredients are released from the oral pouch. In certain aspects, the pouches are transparent or translucent.

Pouches can be formed of one, two, or more materials, and multiple pouches can be used in a single product. Pouch materials can include, but are not limited to, natural or synthetic fabrics incorporating one or more of, e.g., cotton, wool, flax, hemp, ramie, jute, abaca, bamboo, pineapple, wool, silk, wood pulp, other cellulosics, rayon, nylon, polyester, acrylic, microfiber, polypropylene, polymers, et cetera. Pouch materials can be woven or nonwoven; webs of fibers, or contiguous uniform material. Pouch materials can include heat sealable binders, adhesive materials, and/or stitching, which can be incorporated within or separate from a portion of a pouch with which it is or they are used. In embodiments one or more pouch materials can be heat treated or subjected to another process to achieve desired performance or experience (e.g., permeability, strength, moldability, softness, biodegradability, mouthfeel). In particular embodiments, the pouch is made of a woven fiber with having a warp and weave with interstices and pores that cause the pouch to be transparent or translucent.

The fibers may be formed into a woven material. Woven materials may be formed by weaving, knitting, or otherwise interlacing fibers or yarns in a regular, repeating arrangement, to form a fabric material.

In some embodiments, the pouch may be made with a woven material such as a one-layer woven material having one type of pore (e.g., plain and twill weave), two types of pores (e.g., twill weave), three types of pores, or four or more types of pores. Typically, the pores may have a rectangular shape and may differ in dimension, length and positioning of their minimal diameter. The number, size, and distribution of pores may be chosen such that the fabric material is transparent or translucent to light (e.g., such that more than 50% or more than 60% or more than 70% or more than 80% or more than 90% of white light is transmitted through the woven material. In some embodiments, the particles have a D90 particle size greater than the minimum pore dimension.

The porosity of the woven material may be represented by the surface area covered by the pores as a proportion of the total surface area. For example, the percentage surface area due to pores may be greater than 5% or greater than 10% or greater than 15% or greater than 20% or greater than 25% or greater than 30% or greater than 35% or greater than 40% or greater than 50% or greater than 55% or greater than 60% or greater than 65% or greater than 70%.

In some embodiments, the thickness of the woven material comprising the pouch is greater than 15 μm, and may be greater than 20, 35, 50, 80, 100, 120, 150, 175, 18 or 200 μm. The pouch material may, in some embodiments, have a thickness of less than 500, 450, 400, 350, 300, 250, 200, 150, or 100 μm. In some embodiments, the woven material may have a thickness in the range of between 50 and 150 μm, in some implementations preferably between 70 and 100 μm.

Typically, the plurality of particles may be formed by mixing the dry materials, spraying a liquid composition onto the mixed dry materials, optionally continuing mixing following spraying, extruding the sprayed mixture, spheronizing the extruded sprayed mixture, and optionally heating the spheronized extrudate. Extruding may be performed in a suitable extruder. The mixture (e.g., sprayed mixture) may be extruded through a die having from 0.3-2 mm holes (e.g., from 0.5-1.5 mm, from 1-1.5 mm)). The extruder may be operated at from 10-1800 rpm (e.g., 10-600 rpm). Although the openings of the die device may have cross-sections of any desired shape, e.g. circular, oval, square etc., in certain embodiments, the die device comprises openings with substantially circular cross-section and diameters in the range of 0.1 to 1.3 mm. The die may also have multiple size openings such as a first set of openings can having a first diameter in the range of 0.07 to 0.7 mm, such as in the range of 0.15 to 0.6 mm, and suitably in the range of 0.2 to 0.5 mm. A second set of openings can have a second diameter larger than said first diameter. The second diameter is conveniently in the range of 0.4 to 1.3 mm, such as in the range of 0.7 to 1.2 mm.

### Examples

The following examples illustrate specific aspects of the instant description. The examples should not be construed as limiting, as the example merely provides specific understanding and practice of the embodiments and its various aspects.

### Example 1: Exemplary Method of Manufacture

Dry ingredients (including an optional pigment if a color is desired) to form a plurality of particles (e.g., a first plurality of particles, a second plurality of particles) may be mixed in a high-shear granulator such as a GMX 10 (10-liter size) or GMX 150 (150 liter size) produced by Freund Vector. A 150-liter system may appropriately mix 25 kg of dry material. After dry blending for, for example, 1 minute, a water/solvent/active solution (e.g., water/solvent/nicotine such as water/solvent/nicotine base) may be sprayed onto the dry powder mixture to wet the powder and evenly distribute the active. A peristaltic pump may be used to pump the liquid solution from a holding vessel into the granulator while the granulator is mixing. Nicotine, when used as an active, may be supplied in a 40% by weight stock solution in glycerol or propylene glycol. However, this concentration can be either increased or decreased depending on processing conditions and desired final active concentration. Over the course of 3-4 minutes, the water/solvent/active solution may be sprayed onto the mixture. Following spraying, the mixer may be allowed to mix for an additional time period (e.g., 30 seconds).

After mixing in the granulator, the powder may be transferred to the extruder. Before extruding, the appropriate die size may be selected to, for example, control the ultimate extruded particle size. For example, using the extrusion compositions of the present disclosure (e.g., powders comprising filler, low viscosity polymer and high viscosity polymer) a die with 1.2 mm holes allowed for screening for beads with a finished mesh size of between 14-20 (through a 14 mesh and retained on a 20 mesh). For smaller beads (18-35 mesh—through an 18 mesh, retained on a 35 mesh), a die with 0.8 mm size holes was most optimal for screening beads. Appropriate die sizes may be 0.5 mm, 0.6 mm, 0.7 mm, 0.8 mm, 0.9 mm, 1.0 mm, 1.2 mm, and 1.5 mm. In some embodiments, the pouch comprises a first plurality of particles having a first particle size (e.g., particles that pass through a 14 mesh and collected on a 20 mesh) and a second plurality of particles having a second particle size, wherein the second particle size is smaller than the first particle size (e.g., particles that pass through an 18 mesh and collected on a 35 mesh). In some embodiments, the first plurality of particles has a larger particles, e.g., as produced from a 1.22 mm die (and optionally passed through appropriate meshes) and the second plurality of particles has a smaller particles, e.g., as produced from an 0.7 mm die (and optionally passed through appropriate meshes).

Die size tests were performed on a Fuji Paudal MG-55 designed to extrude approximately 15-20 kg/hr and operating at 10-90 rpm. The MG-55 extruder was operated at 40 rpm. Following the extruding, the extrudates were transferred into the spheronizer (Fuji Paudal QJ-230) to form the spheronized extrudates at 1600 rpm for 3 minutes.

Once the beads are spheronized, they may be transferred to a solid coating pan (lab model: Pellegrini T10; production model: IMA GS 150 or IMA GS 300) that is continuously tumbling with heat. Typically, the coater must have a solid coating pan to prevent beads from falling through perforated coating pan holes. The intake heat may be 250 degrees F. and exhaust heat may be 160-172 degrees F. The beads may take 2-3 hours to dry to less than 3.0% moisture measured on an Ohaus moisture balance. From there, the beads may be screened and then packaged into pouches (e.g., at 500 mg per pouch).

### Example 2: Exemplary Formulation

A formulation having 3 mg nicotine (pouch comprising 500 mg particles having citrus flavor and orange color) was prepared. Table 1 provides the dry components used to prepare these particles.

TABLE 1

| Component | Weight (g) |
|---|---|
| Microcrystalline Cellulose | 279.52 |
| High-viscosity Starch | 3.74 |
| Low-viscosity Starch (e.g., OSA-starch) | 33.74 |
| Disodium Phosphate Anhydrous | 74.99 |
| FD&C Yellow Lake #5 | 0.49 |
| FD&C Red #40 lake | 0.49 |
| Citrus flavor/sweetener system | 99.99 |

The dry components in Table 1 were mixed for 1 minute. A solution of reverse-osmosis water (284.99 g) and 40% stock solution of nicotine in glycerol (7.97 g) was formed and sprayed onto the dry blended mixture for 3 minutes. Following spraying, the sprayed mixture was mixed for 30 seconds.

The wetted powder was transferred to the extruder and extruded with a 0.8 mm dome die at 40 rpm. The extrudates were transferred to the spheronizer running at 1600 rpm for 3 minutes. After 3 minutes, the spheronized extrudate was removed from the spheronizer. The wet spherical beads were transferred to a hot tumbler coater and dried for 2-3 hours until the beads are less than 3% moisture as measured on a moisture balance. The beads were screened through an 18 mesh screen with the bottom screen size of 35 mesh.

A pack 500 mg of beads was placed into a pouch that is approximately 40 mm long by sealing one side of the pouch, adding the beads, and sealing the other side of the pouch.

Each pouch contained 3.18 mg nicotine.

### Example 3: Dissolution Studies

Batches of particles comprising microcrystalline cellulose and nicotine were prepared for dissolution studies. Table 2 provides a description of the batches. The low viscosity polymer was a <10 cp modified starch polymer and the high viscosity polymer was a 1500 cp modified starch polymer.

TABLE 2

| | | | High Viscosity | Low Viscosity |
|---|---|---|---|---|
| | Nicotine in | Flavor (wt. | polymer (wt | Polymer (wt |
| Batch # | pouch (g) | percent) | %) | %) |
| 1 | 12 | Citrus (15%) | 6% | |
| 2 | 6 | Citrus (15%) | | 7.5% |
| 3* | 6 | Citrus (15%) | 10% | |
| 6 | 6 | Citrus (15%) | 0.75% | 6.75% |
| 6B | 6 | Citrus (20%) | 0.75% | 6.75% |
| 7 | 6 | Citrus (15%) | 0.375% | 7.125% |
| 8 | 6 | Citrus (15%) | 1.5% | 6.0% |
| 9 | 9 | Citrus (20%) | 0.75% | 6.75% |
| 10 | 3 | Citrus (20%) | 0.75% | 6.75% |
| 11 | 3 | Wintergreen | 0.75% | 6.75% |
| | | (20%) | | |
| 12 | 3 | Wintergreen | 0.75% | 6.75% |
| | | (20%) | | |
| 13^{†} | 3 | Watermelon | 0.75% | 6.75% |
| | | (20%) | | |

*Batch 3 was prepared with natural starch substitution of microcrystalline cellulose. The product resulted in low total release of product and was considered unviable.
^{†}Batch 13 was passed through a 20 mesh screen and collected on a 30 mesh screen.

Dissolution tests were performed on the spheronized batches added to 500 mL of H₂O on a USP Apparatus 2 Paddle operating at 40 RPM at 37° C. 5 mL samples were obtained at 1, 5, 10, 15, 20, 25, 30, and 60 minutes following addition of batches to H₂O dissolution media. Absorbance at 260 nm was measured on each 5 mL sample. Dissolution testing was also performed on the contents of 6 mg or 3 mg ZYN® pouch

Table 3 provides the dissolution data measured for each Batch and ZYN® product. Table 3 also provides the f2 similarity score as compared to the ZYN (6 mg) product. The ZYN® product may have comprised fillers (maltitol and microcrystalline cellulose), a stabilizer (hydroxypropyl cellulose), pH adjusters (sodium carbonate and sodium bicarbonate), a nicotine salt, food grade flavorings, and a sweetener (acesulfame K).

TABLE 3

| | | | | | | | | | f2 |
|---|---|---|---|---|---|---|---|---|---|
| Sample | 1 Min | 5 Min | 10 Min | 15 Min | 20 Min | 25 Min | 30 Min | 60 Min | Value |
| ZYN (6 mg) | 20.7 | 48.9 | 69.2 | 82.1 | 91.1 | 95.7 | 98.0 | 101.1 | |
| ZYN (3 mg) | 25.3 | 48.9 | 67.2 | 79.2 | 85.0 | 89.6 | 92.9 | 95.5 | 67 |
| Batch 1 (12 mg) | 13.3 | 47.8 | 67.5 | 73.6 | 75.2 | 76.0 | 76.5 | 77.4 | 41 |
| Batch 2 (6 mg) | 25.6 | 65.7 | 86.8 | 93.6 | 96.5 | 97.5 | 98.5 | 102.2 | 50 |
| Batch 3 (6 mg) | 6.8 | 30.5 | 52.0 | 61.5 | 66.2 | 69.4 | 71.0 | 75.4 | 33 |
| Batch 6 (6 mg) | 16.4 | 63.5 | 86.6 | 92.0 | 95.0 | 96.4 | 96.8 | 99.6 | 52 |
| Batch 7 (6 mg) | 20.3 | 65.3 | 81.6 | 87.7 | 89.6 | 91.0 | 91.4 | 94.3 | 54 |
| Batch 8 (6 mg) | 9.2 | 55.7 | 80.4 | 89.3 | 91.8 | 95.2 | 95.0 | 95.8 | 57 |
| Batch 9 (9 mg) | 15.6 | 61.5 | 81.7 | 88.6 | 91.9 | 91.2 | 93.9 | 94.6 | 65 |
| Batch 10 (3 mg) | 19.4 | 47.2 | 67.8 | 79.7 | 83.6 | 85.7 | 88.2 | 89.2 | 57 |
| Batch 6B (6 mg) | 10.3 | 47.0 | 74.3 | 85.6 | 90.3 | 93.1 | 94.4 | 97.0 | 65 |
| Batch 12 (3 mg) | 15.0 | 57.6 | 81.8 | 92.9 | 94.2 | 95.5 | 96.4 | 96.7 | 57 |
| Batch 13 (3 mg) | 5.6 | 70.7 | 89.9 | 92.2 | 93.9 | 91.6 | 93.1 | 102.5 | 45 |
| (Small 20-30) | | | | | | | | | |
| Batch 13 (3 mg) | 1.5 | 39.2 | 64.5 | 77.1 | 82.2 | 87.5 | 86.4 | 98.4 | 50 |
| (Large 14-20) | | | | | | | | | |
| Batch 23/24/25 (3 | 22.9 | 69.0 | 89.2 | 96.6 | 98.9 | 100.3 | 100.8 | 100.4 | 46 |
| mg) | | | | | | | | | |
| Batch 27 (3 mg) | 19.4 | 46.5 | 64.0 | 70.3 | 74.8 | 78.0 | 80.3 | 85.6 | 45 |

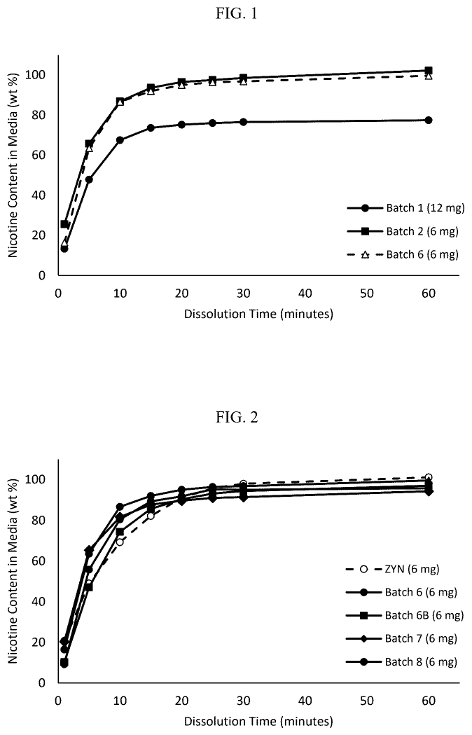

FIG. 1 compares the dissolution of Batches 1 and 2 which vary the nicotine content and contain either high viscosity polymer (Batch 1) or the low viscosity polymer (Batch 2), and Batch 6 which includes a combination of high viscosity polymer and low viscosity polymer. As can be seen, the use of different viscosity polymers and significantly alter the release characteristics of the spheres. For example, addition of a combination of high viscosity polymer and low viscosity polymer in Batch 6 alters the release rate of active in an unexpected manner such that a 6 mg pouch exhibits release similar to a 12 mg pouch with high viscosity polymer.

FIG. 2 provides the release profiles for Batches 6, 6B, 7, and 8 as compared to the ZYN® (6 mg) product. As can be seen, the Batches have a quicker release profile than the ZYN® comparative product.

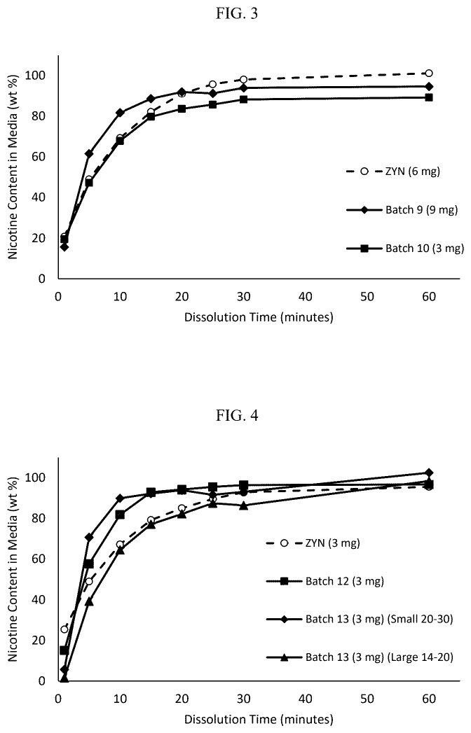

FIG. 3 compares Batches 9 and 10 to the ZYN® product.

FIG. 4 provides a comparison of Batches 12 and 13 (separated by size) with the 3 mg ZYN® product. Batch 12 therefore includes at least two pluralities of particles (the individual release profiles which can be seen as Large Batch 13 and Small Batch 13). As can be seen, particle size has an effect on the release profile and the total release profile of a product can be altered by using different pluralities of particles in a single system.

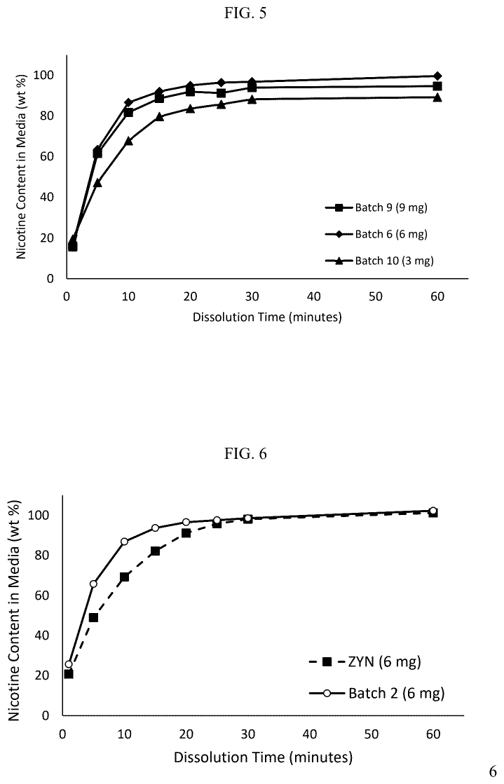

FIG. 5 provides a comparison of the release profiles as varied by active concentration (Batches 6, 9, and 10).

FIG. 6 provides a comparison of the release profiles of Batch 2 (which contained low viscosity polymer and no high viscosity polymer) and the ZYN® product. As can be seen, incorporation of the low viscosity polymers increases the release rate as compared to the ZYN® product.

As various changes can be made in the above-described subject matter without departing from the scope and spirit of the present disclosure, it is intended that all subject matter contained in the above description, or defined in the appended claims, be interpreted as descriptive and illustrative of the present disclosure. Many modifications and variations of the present disclosure are possible in light of the above teachings. Accordingly, the present description is intended to embrace all such alternatives, modifications and variances which fall within the scope of the appended claims.

All documents cited or referenced herein and all documents cited or referenced in the herein cited documents, together with any manufacturer's instructions, descriptions, product specifications, and product sheets for any products mentioned herein or in any document incorporated by reference herein, are hereby incorporated by reference, and may be employed in the practice of the disclosure.

1. A system comprising:
a) pouch; and
b) a plurality of particles contained within the pouch;
wherein, the particles are an optionally coated core, and the core comprises an active, a filler, a buffer, and a low viscosity polymer; and
the active is nicotine, wherein the nicotine is free nicotine or salts thereof, or part of an inclusion complex or ion exchange resin,
the low viscosity polymer is a combination of OSA-starch and hydroxypropyl methyl cellulose (HPMC),
the binder is microcrystalline cellulose, and
the buffer is disodium phosphate, sodium carbonate, trisodium citrate, sodium bicarbonate, or a combination thereof.

2. (canceled)

3. The system according to claim 1, wherein the plurality of particles comprises a first plurality of particles and a second plurality of particles, wherein the first and second pluralities of particles have at least one characteristic distinct from one another.

4-9. (canceled)

10. The system according to claim 3, wherein the first plurality of particles and the second plurality of particles comprise the same low viscosity polymer.

11-21. (canceled)

22. The system according to claim 1, wherein more than 95% of all the particles in the plurality of particles are particles comprising;
a) 30%-80% microcrystalline cellulose by weight;
b) optionally 5%-30% flavorant and/or sweetener by weight;
c) 1%-20% buffer by weight, when the buffer is selected from disodium phosphate, sodium carbonate, sodium bicarbonate, trisodium citrate, trisodium phosphate, and combinations thereof;
d) 1%-10% low viscosity polymer, wherein the low viscosity polymer is the combination of octenyl succinic anhydride (OSA)-starch and hydroxypropyl methyl cellulose (HPMC) combinations thereof) by weight;
and
e) 0.1-10% nicotine by weight, wherein the nicotine is part of a β-cyclodextrin complex or polacrilex.

23-25. (canceled)

26. The system according to claim 1, wherein the pouch is transparent or translucent.

27-44. (canceled)

45. A method comprising;
a) mixing a binder, a low viscosity polymer and, optionally, a colorant and/or high viscosity polymer to form a mixture;
b) spraying a liquid composition onto the mixture or during a portion of the mixing step;
c) extruding the sprayed mixture to form an extrudate;
d) spheronizing the extrudate to form a spheronized extrudate;
wherein an active is mixed in step a) or present in the liquid composition of step b), and
the active nicotine, wherein the nicotine is free nicotine or salts thereof, or part of an inclusion complex or ion exchange resin.

46. The method according to claim 45, wherein further comprising
e) heating the spheronized extrudate.

47. (canceled)

48. The method according to claim 45, wherein the liquid composition comprises the active dissolved in an propylene glycol, glycerol, water, or a combination thereof.

49-52. (canceled)

53. The method according to claim 45, further comprising mixing the spheronized extrudate with a second spheronized extrudate having at least one characteristic distinct from the spheronized extrudate to form a mixed spheronized extrudate and optionally placing the mixed spheronized extrudate in a pouch.

54. The method according to claim 53, wherein the method comprises forming the second spheronized extrudate.

55. The method according to claim 54 wherein forming the second spheronized extrudate comprises
a1) mixing the binder, the low viscosity polymer and a colorant and/or a flavorant and/or a sweetener to form a second mixture;
b1) spraying a second liquid composition comprising an active onto the second mixture or during a portion of the mixing step;
c1) extruding the sprayed mixture to form a second extrudate;
d1) spheronizing the extrudate to form the second spheronized extrudate.

56-63. (canceled)

64. The method according to claim 45, wherein the active is mixed in step a) and the active is nicotine in the form of β-cyclodextrin/nicotine.

65. The system according to claim 26, wherein the pouch is made with a woven fiber having a warp and weave with interstices and pores that cause the pouch to be transparent or translucent.

66. The system according to claim 65, wherein the plurality of particles have a D₉₀ particle size greater than the minimum pore dimension.

67. The system according to claim 65, wherein the plurality of particles have an average particle size of from 200 μm to 10 mm.

68. The system according to claim 1, wherein the weight ratio of OSA-starch:HPMC is from 100:1 to 1:1.

69. The system according to claim 3, wherein the characteristic difference is flavor.
